(19) United States
(12) Patent Application Publication
(10) Pub. No.: US 2008/0039633 A1
(43) Pub. Date: Feb. 14, 2008

(54) Process for preparing arylamines

(76) Inventors: Joerg JUNG, Floersheim (DE); Andreas Meudt, Hofheim (DE); Bernd Wilheilm Lehnemann, Frankfurt am Main (DE)

Correspondence Address: Att: Klaus Schweitzer;ProPat, L.L.C., 425-C South Sharon Amity Road, Charlotte, NC, 28211, US

(21) Appl. No.: 11/836,440

(22) Filed: Aug. 9, 2007

(30) Foreign Application Priority Data
Aug. 10, 2006 (DE) 10 2006 037 399.5

(51) Int. Cl. C07D 211/98; C07C 209/58; C07C 209/72

(52) U.S. Cl. 546/304; 564/395; 564/414

(57) ABSTRACT

The invention relates to a process for preparing arylamines or heteroarylamines or arylamides or heteroarylamides by cross-coupling of primary or secondary amines or amides with substituted aryl or heteroaryl compounds in the presence of a Brønsted base and a catalyst or precatalyst, wherein the catalyst comprises
a) a transition metal, a complex, a salt or a compound of this transition metal selected from the group consisting of Ni, Pd and
b) at least one ligand selected from the group consisting of bidentate bis(phosphino)alkanediyls having the following formula in a solvent or solvent mixture,

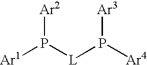

where the radicals Ar¹⁻⁴ are each, independently of one another, an aryl or heteroaryl substituent selected from the group consisting of phenyl, naphthyl, pyridyl and biphenyl or Ar¹⁻⁴ is hydrogen, C₁-, C₂-alkyl, straight-chain, branched or cyclic C₃-C₈-alkyl, and
L is an alkanediyl bridge which has from 1 to 20 carbon atoms.

### CROSS-REFERENCE TO RELATED APPLICATIONS

This application claims priority to German Patent Application 10 2006 037 399.5 filed Aug. 10, 2006 which is hereby incorporated herein by reference in its entirety.

### FIELD OF THE INVENTION

The invention relates to a process for preparing arylamines or heteroarylamines or arylamides or heteroarylamides by cross-coupling of primary or secondary amines or amides with substituted aryl or heteroaryl compounds in the presence of a Brønsted base and a catalyst or precatalyst.

### BACKGROUND OF THE INVENTION

Aryl- and heteroaryl-substituted alkylamines/arylamines or alkylamides/arylamides having various substituents on the nitrogen are important and extremely versatile intermediates in organic synthesis, especially when further functional groups are present in the molecule. Their importance in modern organic synthesis is restricted only by limitations in respect of the availability of this class of compounds.

The standard process for preparing aryl- and heteroaryl-substituted alkylamines/arylamines is the Goldberg reaction which usually requires very high temperatures in order to proceed to completion. However, these generally drastic reaction conditions are rarely tolerated by functional groups and reactive heteroaromatics and can be applied only with great difficulty, if at all, to electron-poor aromatics and in addition are difficult to control.

Standard processes for preparing aryl- and heteroaryl-substituted alkylamides/arylamides usually start out from amines which are often prepared by reduction of nitro compounds. However, this route is often barred for safety reasons or selectivity reasons for applications on an industrial scale, in particular for heteroaromatics such as pyridine.

The processes known at present for preparing aryl- and heteroaryl-substituted alkylamines/arylamines or alkylamides/arylamides thus all still have process engineering or economic disadvantages which sometimes considerably restrict the range of use. Factors of particular importance are especially the high prices of the ligands used and the often poor availability of relatively large amounts of ligands which make use in industrial applications difficult or impossible. In addition, many ligands, in particular those from the class of trialkylphosphines, can be handled only with strict exclusion of air in order to avoid oxidative decomposition or even spontaneous ignition, which makes use on a relatively large scale significantly more difficult and expensive.

It would be very desirable to have a process which can convert substituted alkylamines, arylamines, alkylamides or arylamides and haloaromatics or haloheteroaromatics into the corresponding aryl- or heteroaryl-substituted alkylamines/arylamines or alkylamideslarylamides and at the same time gives high yields and makes do with cheap and readily available and easy-to-handle ligands. As mentioned above, the synthetic methods published hitherto for this purpose do not satisfactorily solve this problem, as will be demonstrated further with the aid of a few examples:
use of expensive ligands (e.g. P^{t}Bu₃, Hartwig et al., U.S. Pat. No. 6,100,398) and complicated isolation of the product by chromatography
use of air-sensitive ligands (e.g. P^{t}Bu₃, Hartwig et al., U.S. Pat. No. 6,100,398)
use of ligands which are difficult to synthesize (ferrocene-based ligands, Hartwig et al., WO 0211883)
complicated isolation of the product by chromatography
complicated or difficult, often multistage ligand syntheses (Buchwald et al., WO 0002887), complicated isolation of the product by chromatography
deactivation of the metal-ligand complexes during the reaction, often no opportunity of restarting the reaction by addition of further catalyst and therefore loss of entire batches. This phenomenon can be explained by low stability of the complexes of metal and monodentate ligands and by the high oxidation sensitivity of the ligands.

### SUMMARY OF ADVANTAGEOUS EMBODIMENTS OF THE INVENTION

The present process solves all these problems and provides a process for preparing arylamines and heteroarylamines, aryl- or heteroaryl-substituted alkylamides/arylamides by cross-coupling of primary or secondary alkylamines or arylamines or of primary or secondary alkylamides or arylamides with substituted aryl or heteroaryl compounds (I) in the presence of a Brønsted base and a catalyst or precatalyst comprising

a.) a transition metal, a complex, salt or compound of this transition metal selected from the group consisting of Ni, Pd and
b.) at least one ligand selected from the group consisting of bidentate bis(phosphino)alkanediyls having the formula:

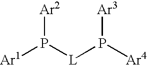

The radicals Ar¹⁻⁴ are each, independently of one another, an aryl or heteroaryl substituent selected from the group consisting of phenyl, naphthyl, pyridyl, biphenyl and the like in which hydrogen may have been replaced by other radicals such as lower alkyl substituents, halogen atoms, sulfonic acid groups, carboxylic acid groups, lower alkyloxy substituents or the like or Ar¹⁻⁴ is hydrogen, C₁-, C₂-alkyl, straight-chain, branched or cyclic C₃-C₈-alkyl which may be monosubstituted or polysubstituted by Cl, Br, I, OH, NH₂, NO₂, CN, COOH, lower alkylamino, lower alkyldiamino, lower alkyloxy or lower alkyloxycarbonyl or lower alkylcarbonyloxy, where lower alkyl is hereinafter a C₁-C₄-alkyl radical, preferably methyl or ethyl.

L is an alkanediyl bridge which has from 1 to 20 carbon atoms and can be either linear or branched. L is preferably an alkanediyl bridge selected from the group consisting of ethane-1,2-diyl, propane-1,3-diyl, butane-1,4-diyl and 2,2-dimethylpropane-1,3-diyl.

Some of the abovementioned chelates are known; but they have hitherto predominantly been used in C,C couplings, first and foremost in couplings of Grignard compounds with aryl halides in the presence of such ligands and Ni salts as catalysts. It has surprisingly been found that the abovementioned ligands in combination with a transition metal, a complex, salt or compound of this transition metal selected from the group consisting of Ni, Pd are suitable for catalytic C,N bond formation.

The reaction is typically carried out in a solvent or solvent mixture. As solvent or constituent of the solvent mixture, it is possible to use any solvent which is compatible with the reactants but preferably ether solvents (e.g. dioxane, THF, 1,2-dimethoxyethane, monoglyme, diglyme or higher glymes) or aromatics (e.g. benzene, toluene, xylene, trimethylbenzenes, ethylbenzene) or alcohols (isopropanol, ethanol, 2-methoxyethane, 1-methoxy-2-propanol, glycol) or amides (e.g. DMF, NMP).

The process of the invention has the following advantages:
the ligands are commercially available at an economically attractive price and in large quantities, in particular when all substituents Ar¹⁻⁴ are phenyl radicals.
“Fine tuning” of the ligands can be achieved relatively easily by appropriate selection of the alkanediyl linker. In particular, the “bite angle” of the two phosphine subunits can be adapted so that optimal reactivity results. Thus, for example, the “bite angle” of the ligand in which L=propanediyl obviously represents an optimum compared to ethanediyl and butanediyl in the synthesis of 2,2-dimethyl-N-pyridin-2-yl-propionamide, as can be seen from comparison of the yields (cf. examples 10, 11 and 12).
Apart from the attractive economic properties, this class of ligands displays a high stability toward air and moisture, so that handling is considerably simplified compared to other classes of ligands, which is of particularly great importance on an industrial scale.
The actual active complexes resulting from the transition metal sources and the bidentate ligands are very stable, so that the catalyst is not easily deactivated and the overall catalyst system is therefore very robust.
The process of the invention therefore widens the range of use of the previously known C,N coupling technologies tremendously by means of the above-mentioned parameters which can additionally be finely adjusted.
This class of ligands makes it possible, particularly in heteroaromatic systems, to couple not only amines but also amides, which greatly widens the range of use since this reaction can often not be brought about using other ligand systems.

### DETAILED DESCRIPTION OF ADVANTAGEOUS EMBODIMENTS OF THE INVENTION

Equation 1 below illustrates the course of the synthesis in the process of the invention:

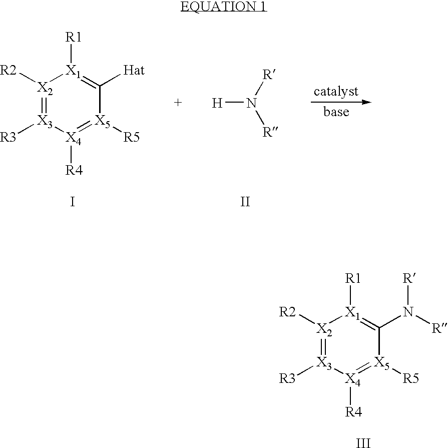

In equation 1 Hal is fluorine, chlorine, bromine, iodine, alkoxy or a sulfonate leaving group such as trifluoromethanesulfonate (triflate), nonafluorobutanesulfonate (nonaflate), methanesulfonate, benzenesulfonate, para-toluenesulfonate.

The atoms X₁₋₅ are each, independently of one another, carbon or the moieties XᵢRᵢ (i=1-5) are nitrogen or two adjacent moieties XᵢRᵢ which are bound to one another by a formal double bond are together 0 (furans), S (thiophenes), NH or NRᵢ (i=1-5) (pyrroles).

Preferred compounds of the formula (I) which can be reacted by the process of the invention are, for example, benzenes, pyridines, pyrimidines, pyrazines, pyridazines, furans, thiophenes, pyrroles, any N-substituted pyrroles or naphthalenes, quinolines, indoles, benzofurans, etc.

The radicals R₁₅ are substituents selected from the group consisting of hydrogen, methyl, ethyl, primary, secondary or tertiary, cyclic or acyclic alkyl radicals which have from 3 to 20 carbon atoms and in which one or more hydrogen atoms may have been replaced by fluorine or chlorine or bromine, e.g. CF₃, hydroxy, alkoxy, amino, alkylamino, dialkylamino, arylamino, diarylamino, alkylarylamino, pentafluorosulfuranyl, phenyl, substituted phenyl, heteroaryl, substituted heteroaryl, thio, alkylthio, arylthio, diarylphosphino, dialkylphosphino, alkylarylphosphino, substituted or unsubstituted aminocarbonyl, COO⁻, alkyl, or aryloxycarbonyl, hydroxyalkyl, alkoxyalkyl, fluorine or chlorine, nitro, cyano, arylsulfone or alkylsulfone, arylsulfonyl or alkylsulfonyl, or two adjacent radicals R₁₅ can together correspond to an aromatic, heteroaromatic or aliphatic fused-on ring.

R′ and R″ can be identical or different and can each be, independently of one another, an alkyl radical selected from the group consisting of hydrogen, C₁-, C₂-alkyl, straight-chain, branched or cyclic C₃-C₂₀-alkyl, substituted or unsubstituted aryl or heteroaryl or an acyl radical selected from the group consisting of formyl, acetyl, linear or branched C₃-C₂₀-acetyl and substituted or unsubstituted aroyl or heteroaroyl or together form a ring. R′ and R″ are preferably not simultaneously hydrogen.

Typical examples of compounds II are thus methylamine, ethylamine, 1-methylethylamine, propylamine, 1-methylpropylamine, 2-methylpropylamine, 1,1-dimethylethylamine, butylamine and pentylamine, cyclopropylamine, cyclobutylamine, cyclopentylamine, cyclohexylamine, phenylamine, benzylamine, morpholin from the group of amines or acetamide, benzamide, 2,2-dimethylpropionamide from the group of amides.

According to the invention, a transition metal or a salt, a complex or a metal-organic compound of a transition metal selected from the group consisting of Ni, Pd, preferably on a support such as carbon, together with a bidentate bis(phosphino)alkanediyl ligand is used as catalyst. The catalyst can be added in finished form or can form in situ, e.g. from a precatalyst by reduction or hydrolysis or from a transition metal salt and an added ligand by complex formation. The catalyst is used in combination with one or more but at least one bidentate bis(phosphino)alkanediyl ligand. The transition metal can be used in any oxidation state. According to the invention, it is used in a molar ratio to the reactant I of from 0.0001 to 100, preferably from 0.01 to 10, particularly preferably from 0.01 to 2.

Preference is given to ligands having the structure shown below

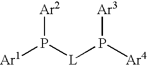

in combination with palladium or nickel as catalyst, where the radicals Ar¹⁻⁴ are each, independently of one another, an aryl or heteroaryl substituent selected from the group consisting of phenyl, naphthyl, pyridyl, biphenyl and the like in which hydrogen may have been replaced by other radicals such as lower alkyl substituents, halogen atoms, sulfonic acid groups, carboxylic acid groups, lower alkyloxy substituents or the like.

L has the meanings indicated above for the structure depicted.

The addition of Brønsted bases to the reaction mixture is necessary to achieve acceptable reaction rates. Well-suited bases are, for example, hydroxides, alkoxides and fluorides of the alkali metals and alkaline earth metals, carbonates, hydrogencarbonates and phosphates of the alkali metals and mixtures thereof. Particularly useful bases are the bases of the group potassium tert-butoxide, sodium tert-butoxide, cesium tert-butoxide, lithium tert-butoxide and the corresponding isopropoxides for the coupling of amides and the bases of the groups sodium carbonate, potassium carbonate, cesium carbonate, potassium phosphate for the coupling of amides. It is usual to use at least the molar amount of base which corresponds to the molar amount of the amine or amide to be coupled, mostly from 1.0 to 6 equivalents, preferably from 1.2 to 3 equivalents, of base based on the compound (II).

The reaction is carried out in a suitable solvent or a single-phase or multiphase solvent mixture which has a sufficient solvent capability for all participating reactants, with heterogeneous reactions also being possible (e.g. use of virtually insoluble bases). The reaction is preferably carried out in polar, aprotic or protic solvents. Well-suited solvents are open-chain and cyclic ethers and diethers, oligoethers and polyethers and also substituted simple or multiple alcohols and substituted or unsubstituted aromatics. Particular preference is given to using a solvent or mixture of a plurality of solvents selected from the group consisting of diglyme, substituted glymes, 1,4-dioxane, isopropanol, tert-butanol, 2,2-dimethyl-1-propanol, toluene, xylene.

The reaction can be carried out at temperatures in the range from room temperature to the boiling point of the solvent used and the pressure used. To achieve a more rapid reaction, preference is given to carrying it out at elevated temperatures in the range from 0 to 240° C. Particular preference is given to the temperature range from 20 to 200° C., in particular from 50 to 150° C.

The concentration of the reactants can be varied within a wide range. The reaction is advantageously carried out at a very high concentration, with the solubilities of the reactants and reagents in the respective reaction medium having to be taken into account. The reaction is preferably carried out in the range from 0.05 to 5 mol/l based on the reactants present in a substoichiometric amount (depending on the relative prices of the reactants).

Amine or amide and aromatic or heteroaromatic reactant (I) can be used in a molar ratio of from 10:1 to 1:10, preferably from 3:1 to 1:3 and particularly preferably from 1.2:1 to 1:1.2.

In a preferred embodiment, all materials are placed in the reaction vessel and the mixture is heated to the reaction temperature while stirring. In a further preferred embodiment, which is particularly suitable for use on a large scale, the compound (II) and, if appropriate, further reactants, e.g. base and catalyst or precatalyst, are metered into the reaction mixture during the reaction. As an alternative, the reaction can also be carried out in an addition-controlled fashion by slow addition of the base. The selectivities are, according to the invention, very high and it is usually possible to find conditions under which no further by-products apart from very small amounts of dehalogenation product can be detected.

The work-up is usually carried out, after removal of inorganic salts by means of water, by customary methods, i.e. in the laboratory by chromatography and in industry by distillation or recrystallization.

The process of the invention is illustrated by the following examples without the invention being restricted thereto:

### EXAMPLE 1

### Coupling of 3-methylpiperidine with 4-bromobenzotrlfluoride(4-bromotrifluoromethylbenzene) (catalyst: Pd(OAc)₂/2,2-dimethyl-1,3-bis(di-phenylphosphino)propane)

5.8 g of sodium t-butoxide (60.1 mmol), 5.2 g of 3-methylpiperidine (52.6 mmol) and 8.5 g of 4-bromobenzotrifluoride (37.6 mmol) are dissolved or suspended in 50 ml of dioxane and admixed at 80° C. with a suspension of 0.167 g of palladium(II) acetate (2 mol %) and 0.43 g of 2,2-dimethyl-1,3-bis(diphenylphosphino)propane (2.5 mol %). The mixture is subsequently refluxed and the conversion is monitored by HPLC. After about 4 hours, the conversion is >98%. The work-up is carried out by addition of water to dissolve the precipitated salts, addition of toluene and phase separation. The upper, product-containing phase is evaporated on a rotary evaporator and the product is purified by chromatography. This gives 7.5 g (82%) of coupling product (3-methyl-1-(4-trifluoromethylphenyl)piperidine).

### EXAMPLE 2

### Coupling of 3-methylpiperidine with 4-bromobenzotrifluoride (catalyst: Pd(dba)₂/2,2-dimethyl-1,3-bis(diphenylphosphino)propane)

As example 1 but using 0.40 g of bis(dibenzylideneacetone)palladium(0) instead of 0.167 g of palladium(II) acetate. As in example 1, the reaction was concluded after a short reaction time (in this case boiling overnight). Yield: 7.8 g (84%)

### EXAMPLE 3

### Coupling of 3-methylpiperidine with 4-chlorobenzotdifluoride (catalyst: Pd(OAc)₂/2,2-dimethyl-1,3-bis(diphenylphosphino)propane)

As example 1 but using 6.7 g of 4-chlorobenzotrifluoride (37.6 mmol) instead of 8.5 g of 4-bromobenzotrifluoride (37.6 mmol). To achieve complete conversion (>95%), boiling had to be continued for a somewhat long time (60 h) when using the less reactive chloro compound. However, the yield was comparable with that in the two previous examples (7.1 g, 78%).

### EXAMPLE 4

### Coupling of 2-chloroaniline with 4-bromoanisole (catalyst: Pd(OAc)₂/2,2-dimethyl-1,3-bis(diphenylphosphino)propane)

5.9 g of sodium t-butoxide (61.3 mmol), 6.7 g of 2-chloroaniline (52.6 mmol) and 7.0 g of 4-bromoanisole (37.6 mmol) are dissolved or suspended in 50 ml of dioxane and admixed at 80° C. with a suspension of 0.167 g of palladium(II) acetate (2 mol %) and 0.43 g of 2,2-dimethyl-1,3-bis(diphenylphosphino)propane (2.5 mol %). The mixture is subsequently refluxed and the conversion is monitored by HPLC. The conversion is quantitative (>95%) after 72 hours. The work-up is carried out by addition of water to dissolve the precipitated salts, addition of toluene and phase separation. The upper, product-containing phase is evaporated on a rotary evaporator and the product is purified by chromatography. This gave 6.4 g (73%) of coupling product (2-chlorophenyl)(4-methoxyphenyl)amine.

### EXAMPLE 5

### Coupling of 2-chloroaniline with 4-bromobenzotrifluoride (catalyst: Pd(OAc)₂/2,2-dimethyl-1,3-bis(diphenylphosphino)propane)

4.8 g of sodium t-butoxide (48.4 mmol), 4.1 g of 2-chloroaniline (33.2 mmol) and 7.0 g of 4-bromobenzotrifluoride (30.2 mmol) are dissolved or suspended in 50 ml of dioxane and admixed at 80° C. with a suspension of 0.14 g of palladium(1) acetate (2 mol %) and 0.33 g of 2,2-dimethyl-1,3-bis(diphenylphosphino)propane (2.5 mol %). The mixture is subsequently refluxed and the conversion is monitored by HPLC. The conversion is quantitative (>95%) after 72 hours. The work-up is carried out by addition of water to dissolve the precipitated salts, addition of toluene and phase separation. The upper, product-containing phase is evaporated on a rotary evaporator and the product is purified by chromatography. This gave 5.3 g (64%) of coupling product (2-chloro-phenyl)(4-trifluoromethylphenyl)amine.

### EXAMPLE 6

### Coupling of 3-methylpiperidine with 4-bromobenzotrifluoride (catalyst: Pd(OAc)₂/1,4-bis(diphenylphosphino)butane)

2.9 g of sodium t-butoxide (30.1 mmol), 2.6 g of 3-methylpiperidine (26.3 mmol) and 4.2 g of 4-bromobenzotrifluoride (18.8 mmol) are dissolved or suspended in 25 ml of dioxane and admixed at 80° C. with a suspension of 0.088 g of palladium(II) acetate (2 mol %) and 0.201 g of 1,4-bis(diphenylphosphino)butane (2.5 mol %). The mixture is subsequently refluxed and the conversion is monitored by HPLC. After about 5 hours, the conversion is >98%. The work-up is carried out by addition of water to dissolve the precipitated salts, addition of toluene and phase separation. The upper, product-containing phase is evaporated on a rotary evaporator and the product is purified by chromatography. This gave 3.1 g (87%) of coupling product (3-methyl-1-(4-trifluoromethylphenyl)piperidine).

### EXAMPLE 7

### Coupling of 3-methylpiperidine with 4-bromobenzotrifluoride (catalyst: Pd(OAc)₂/1,3-bis(diphenylphosphino)propane)

2.9 g of sodium t-butoxide (30.1 mmol), 2.6 g of 3-methylpiperidine (26.3 mmol) and 4.2 g of 4-bromobenzotrifluoride (18.8 mmol) are dissolved or suspended in 25 ml of dioxane and admixed at 80° C. with a suspension of 0.088 g of palladium(II) acetate (2 mol %) and 0.194 g of 1,3-bis(diphenylphosphino)propane (2.5 mol %). The mixture is subsequently refluxed and the conversion is monitored by HPLC. After about 5 hours, the conversion is >98%. The work-up is carried out by addition of water to dissolve the precipitated salts, addition of toluene and phase separation. The upper, product-containing phase is evaporated on a rotary evaporator and the product is purified by chromatography. This gave 3.2 g (90%) of coupling product (3-methyl-1-(4-trifluoromethylphenyl)piperidine).

### EXAMPLE 8

### Coupling of 3-methylpiperidine with 4-bromobenzotrifluoride (catalyst: Pd(OAc)₂/1,2-bis(diphenylphosphino)ethane)

2.9 g of sodium t-butoxide (30.1 mmol), 2.6 g of 3-methylpiperidine (26.3 mmol) and 4.2 g of 4-bromobenzotrifluoride (18.8 mmol) are dissolved or suspended in 25 ml of dioxane and admixed at 80° C. with a suspension of 0.088 g of palladium(1) acetate (2 mol %) and 0.187 g of 1,2-bis(diphenylphosphino)ethane (2.5 mol %). The mixture is subsequently refluxed and the conversion is monitored by HPLC. After about 5 hours, the conversion is >98%. The work-up is carried out by addition of water to dissolve the precipitated salts, addition of toluene and phase separation. The upper, product-containing phase is evaporated on a rotary evaporator and the product is purified by chromatography. This gave 2.9 g (82%) of coupling product (3-methyl-1-(4-trifluoromethylphenyl)piperidine).

Apart from the couplings of amines, the systems described are also especially active in the coupling of amides with aromatics, i.e. in particular in the coupling with heteroaromatics such as pyridines. In these couplings, potassium carbonate can advantageously be used as base.

### EXAMPLE 9

### Coupling of 2-chloropyridine with 4-fluorobenzamide (catalyst: Pd(OAc)₂/2,2-dimethyl-1,3-bis(diphenylphosphino)propane)

4.0 g of potassium carbonate (28.9 mmol), 3.5 g of 4-fluorobenzamide (25.3 mmol) and 2.1 g of 2-chloropyridine (18.1 mmol) are dissolved or suspended in 25 ml of dioxane and admixed at 80° C. with a suspension of 0.036 g of palladium(II) acetate (0.9 mol %) and 0.200 g of 1,3-bis(diphenylphosphino)propane (2.5 mol %). The mixture is subsequently refluxed and the conversion is monitored by HPLC. After boiling overnight, the conversion is >98%. The work-up is carried out by addition of water to dissolve the precipitated salts, addition of toluene and phase separation. The upper, product-containing phase is evaporated on a rotary evaporator and the product is purified by chromatography. This gave 3.5 g (89%) of coupling product (4-fluoro-N-pyridin-2-yl-benzamide).

### EXAMPLE 10

### Coupling of 2-chloropyridine with 2,2-dimethylpropionamide (catalyst: Pd(OAc)₂/2,2-dimethyl-1,3-bis(diphenylphosphino)propane)

3.1 g of potassium carbonate (22.7 mmol), 2.0 g of 2,2-dimethylpropionamide (20.0 mmol) and 1.7 g of 2-chloropyridine (14.2 mmol) are dissolved or suspended in 40 ml of dioxane and admixed at 80° C. with a suspension of 0.027 g of palladium(II) acetate (0.9 mol %) and 0.156 g of 2,2-dimethyl-1,3-bis(diphenylphosphino)propane (2.5 mol %). The mixture is subsequently refluxed and the conversion is monitored by HPLC. After boiling overnight, the conversion is >98%. The work-up is carried out by addition of water to dissolve the precipitated salts, addition of toluene and phase separation. The upper, product-containing phase is evaporated on a rotary evaporator and the product is purified by chromatography. This gave 2.6 g (88%) of coupling product (2,2-dimethyl-N-pyridin-2-yl-propionamide)

### EXAMPLE 11

### Coupling of 2-chloropyridine with 2,2-dimethylpropionamide (catalyst: Pd(OAc)₂/1,4-bis(diphenylphosphino)butane)

As example 10, but 0.151 g of 1,4-bis(diphenylphosphino)butane (2.5 mol %) was used instead of 0.156 g of 2,2-dimethyl-1,3-bis(diphenylphosphino)propane (2.5 mol %). Yield: 2.4 g (81%).

### EXAMPLE 12

### Coupling of 2-chloropyridine with 2,2-dimethylpropionamide (catalyst: Pd(OAc)₂/1,2-bis(diphenylphosphino)ethane)

As example 10, but 0.151 g of 1,4-bis(diphenylphosphino)ethane (2.5 mol %) is used instead of 0.141 g of 2,2-dimethyl-1,3-bis(diphenylphosphino)propane (2.5 mol %). Yield: 2.5 g (85%).

### EXAMPLE 13

### Coupling of 2-chloropyridine with Z 2-dimethylpropionamide (catalyst: Pd(OAc)₂/triphenylphosphine; comparative experiment)

As example 10, but 0.187 g of triphenylphosphine (5 mol %) is used instead of 0.141 g of 2,2-dimethyl-1,3-bis(diphenylphosphino)propane (2.5 mol %). However, no conversion was able to be achieved using this ligand.

1. A process for preparing arylamines or heteroarylamines or arylamides or heteroarylamides comprising cross-coupling primary or secondary amines or amides with substituted aryl or heteroaryl compounds in the presence of a Brønsted base and a catalyst or precatalyst, wherein the catalyst comprises
a) a transition metal, a complex, a salt or a compound of this transition metal selected from the group consisting of Ni, Pd and
b) at least one ligand selected from the group consisting of bidentate bis(phosphino)alkanediyls having the following formula in a solvent or solvent mixture, 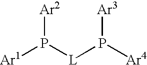
where the radicals Ar¹⁻⁴ are each, independently of one another, an aryl or heteroaryl substituent selected from the group consisting of phenyl, naphthyl, pyridyl and biphenyl in which hydrogen may have been replaced by lower alkyl substituents, halogen atoms, sulfonic acid groups, carboxylic acid groups, lower alkyloxy substituents
or Ar¹⁻⁴ is hydrogen, C₁-, C₂-alkyl, straight-chain, branched or cyclic C₃-C₈-alkyl which may be monosubstituted or polysubstituted by Cl, Br, I, OH, NH₂, NO₂, CN, COOH, lower alkylamino, lower alkyldiamino, lower alkyloxy or lower alkyloxycarbonyl or lower alkylcarbonyloxy, where lower alkyl is a C₁-C₄-alkyl radical, and
L is an alkanediyl bridge which has from 1 to 20 carbon atoms and is either linear or branched.

2. The process as claimed in claim 1, wherein L is an alkanediyl bridge selected from the group consisting of ethane-1,2-diyl, propane-1,3-diyl, butane-1,4-diyl and 2,2-dimethylpropane-1,3-diyl.

3. The process as claimed in claim 1, wherein the substituted aryl or heteroaryl compound is a compound of the formula (I), 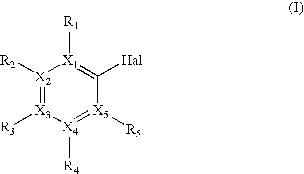
where
Hal is fluorine, chlorine, bromine, iodine, C₁-C₄-alkoxy, trifluoromethanesulfonate, nonafluorotrimethylmethanesulfonate, methanesulfonate, 4-toluenesulfonate, benzenesulfonate, 2-naphthalenesulfonate, 3-nitrobenzenesulfonate, 4-nitrobenzenesulfonate, 4-chlorobenzenesulfonate or 2,4,6-triisopropylbenzenesulfonate and
the radicals R₁₋₅ are identical or different substituents from the group consisting of hydrogen, methyl, ethyl, primary, secondary or tertiary, cyclic or acyclic alkyl radicals which have from 3 to 20 carbon atoms and in which one or more hydrogen atoms are optionally replaced by fluorine or chlorine or bromine, hydroxy, lower alkyloxy, amino, lower alkylamino, di-lower-alkylamino, arylamino, diarylamino, lower-alkylarylamino, pentafluorosulfuranyl, phenyl, substituted phenyl, heteroaryl, substituted heteroaryl, thio, lower alkylthio, arylthio, diarylphosphino, di-lower-alkylphosphino, lower alkylarylphosphino, substituted or unsubstituted aminocarbonyl, CO₂⁻, lower alkylcarbonyl or aryloxycarbonyl, hydroxy-lower-alkyl, lower-alkyloxy-lower-alkyl, fluorine, chlorine, nitro, cyano, arylsulfone or lower alkylsulfone, arylsulfonyl or lower alkylsulfonyl, where lower alkyl is a C₁-C₄-alkyl radical, aryl is phenyl or naphthyl and heteroaryl is pyridinyl, imidazolyl, thienyl or furanyl, or two adjacent radicals R₁₋₅ together correspond to an aromatic, heteroaromatic or aliphatic fused-on ring.

4. The process as claimed in claim 1, wherein the primary or secondary amine or amide is a compound of the formula (II), 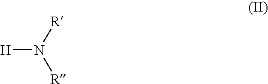
where R′ and R″ are identical or different and are each, independently of one another, a radical selected from the group consisting of hydrogen, methyl, ethyl, linear, branched C₃-C₂₀-alkyl or cyclic C₃-C₂₀-alkyl, substituted or unsubstituted aryl or heteroaryl, where aryl is phenyl or naphthyl and heteroaryl is pyridinyl, imidazolyl, thienyl or furanyl; or an acyl radical selected from the group consisting of formyl, acetyl, linear or branched C₃-C₂₀-acetyl or substituted or unsubstituted aroyl or heteroaroyl, where aroyl is phenylcarbonyl or naphthylcarbonyl and heteroaroyl is pyridinylcarbonyl, imidazolylcarbonyl, thienylcarbonyl or furanylcarbonyl; or together form a ring.

5. The process as claimed in claim 1, wherein the transition metal used for the catalysis is palladium.

6. The process as claimed in claim 5, wherein the palladium source is palladium(II) acetate.

7. The process as claimed in claim 1, wherein the alkanediyl bridge L has a length of from 1 to 4 carbon atoms.

8. The process as claimed in claim 1, wherein from 1.0 to 3 equivalents of Brønsted base based on the substituted aryl or heteroaryl compound is used.

9. The process as claimed in claim 8, wherein the Brønsted base is sodium tert-butoxide.

10. The process as claimed in claim 8, wherein the Brønsted base is potassium carbonate.

11. The process as claimed in claim 1, wherein the substituted aryl or heteroaryl compound is a 2-halopyridine which may be additionally substituted or a 4-halopyridine which may be additionally substituted.

12. The process as claimed in claim 1, wherein hydrocarbons, halogenated hydrocarbons, open-chain or cyclic ethers or diethers, oligoethers or polyethers, tertiary amines, DMSO, NMP, DMF, DMAc and substituted simple or multiple alcohols or substituted or unsubstituted aromatics or a mixture of a plurality of these solvents is/are used as a solvent or solvent mixture.

13. The process as claimed in claim 1, wherein the cross-coupling reaction is carried out at a temperature in the range from 0 to 240° C.

14. The process as claimed in claim 1, wherein the catalyst is used in a molar ratio to the substituted aryl or heteroaryl compound of from 0.001 to 25.
